(19) United States
(12) Patent Application Publication
(10) Pub. No.: US 2007/0208375 A1
(43) Pub. Date: Sep. 6, 2007

(54) Surgical device

(76) Inventors: Kouji Nishizawa, Hitachinaka (JP); Takeshi Hoshino, Kodaira (JP); Yukinobu Maruyama, Kokubunji (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/359,375

(22) Filed: Feb. 23, 2006

(51) Int. Cl. A61B 17/00

(52) U.S. Cl. 606/205

(57) ABSTRACT

The invention provides a surgical device which can control a position and an attitude of a multi degree of freedom type grip portion (forceps) in a dummy manner on the basis of an operation of an operator in an operating portion. In a surgical device provided with a leading end joint portion having a leading end grip portion, a near-side joint portion having an operation portion, a handle portion supporting the operation portion, and an arm portion storing a wire for linking motions of the leading end joint portion and the near-side joint portion, the leading end joint portion is moved downward and upward by operating the operation portion and the handle portion upward and downward around the near-side joint portion, and the leading end joint portion is moved rightward and leftward by operating the operation portion and the handle portion leftward and rightward, thereby making the leading end joint portion execute a swing motion, and the leading end grip portion is opened and closed by opening and closing finger rests of the operation portion.

### BACKGROUND OF THE INVENTION

1. Field of the Invention

The present invention relates to a surgical device used in a clinical field, and more particularly to a surgical device which can apply a dummy operation to multi degrees of freedom type forceps a position and an attitude of which are operated by a wire driving type joint portion.

2. Description of Related Art

As a prior art of a medical manipulator which has a movable portion having two degrees of freedom in a leading end of the forceps, and has a forceps operating portion in a rear end portion of the forceps so as to finely operated the forceps, as shown in patent document 1 (JP-A-2001-276091), there is a structure in which a first rotational joint and a second rotational joint are provided in the leading end of the forceps, a positioning operation of a leading end of the forceps is executed by controlling respective rotational joints via a wire, a gear or the like on the basis of a motor drive in an operation portion, and an opening and closing magnitude of the forceps is controlled by operating a lever provided in an operation rod.

Further, as a prior art of the multi degrees of freedom type treatment device, patent document 2 (JP-A-2004-154164) discloses a structure in which a treatment device main body to which a treatment portion is connected via a joint portion is provided with a joy stick bending the treatment portion in a vertical direction and a lateral direction, a dial rotating the treatment portion, and a lever opening and closing the treatment portion, whereby the treatment portion is easily set to desired position and attitude.

Further, as a prior art of the surgical manipulator, there is disclosed a structure in which a compactness and a controllability of the manipulator are improved by keeping a length and a phase of a drive wire regardless of an angular change of the joint (refer, for example, to patent document 3 (JP-A-2004-122286)).

### BRIEF SUMMARY OF THE INVENTION

However, in the prior art shown in the patent document 1, since the rotational operations of the first rotational shaft and the second rotational shaft provided in the joint portion execute positioning and attitude determination of the forceps by applying complex lateral and vertical operations to a joy stick provided in the operation rod, and the positioning and the attitude determination are executed by driving a motor installed in the operation rod, it is necessary to execute a complex control on the basis of the operation of the joy stick and the drive of the motor.

Further, in the patent document 2, the drive wire is directly operated by the manually operated joy stick without using the motor, however, the operation of the joy stick requires a lot of skill at a time of appropriately setting the position and the attitude of the treatment portion. Further, the patent document 3 is structured such as to execute a swing motion and an opening and closing motion of a blade on the basis of the motor control, and requires the motor drive.

An object of the present invention is to provide a surgical device which can control a position and an attitude of a multi degree of freedom type grip portion (forceps) in a dummy manner on the basis of an operation of an operator in an operating portion.

In order to solve the problem mentioned above, the present invention mainly employs the following structures.

There is provided a surgical device comprising:

a leading end joint portion having a leading end grip portion;

a near-side joint portion having an operation portion;

a handle portion supporting the operation portion; and

an arm portion storing a wire for linking motions of the leading end joint portion and the near-side joint portion,

wherein the leading end joint portion is moved downward and upward by operating the operation portion and the handle portion upward and downward around the near-side joint portion, and the leading end joint portion is moved rightward and leftward by operating the operation portion and the handle portion leftward and rightward, thereby making the leading end joint portion execute a swing motion, and

wherein the leading end grip portion is opened and closed by opening and closing the operation portion.

Further, in the surgical device, a pair of rolling contact means achieving a rolling contact are provided in the leading end joint portion and the near-side joint portion,

wherein a pulley is provided in one rolling contact means of a pair of rolling contact means in the leading end joint portion and the near-side joint portion, the leading end grip portion is attached to the pulley in the leading endjoint portion and the operation portion is attached to the pulley in the near-side joint portion, and a wire is wound around the pulley of the leading end joint portion and the near-side joint portion,

wherein a wire is wound around the other rolling contact means in a pair of rolling contact means in the leading end joint portion and the near-side joint portion,

the leading end joint portion is swung on the basis of a rotational drive via the wire in the other rolling contact means in a pair of rolling contact means and a rotational drive via the wire in the pulley, by moving the operation portion and the handle portion upward and downward, and rightward and leftward, and

wherein the leading end grip portion is opened and closed on the basis of a rotational drive via the wire in the pulley, by opening and closing the operation portion.

In accordance with the-present invention, since the position and the attitude of the grip portion serving as the forceps execute a dummy motion to the operation of the operator in the operation portion apart from the forceps without using any electronic control of an actuator or the like, it is possible to remote control the motion of the-grip portion on the basis of a visceral operation without requiring a lot of skill.

Further, it is possible to provide a surgical device which has a simple structure and is easily operated.

Other objects, features and advantages of the invention will become apparent from the following description of the embodiments of the invention taken in conjunction with the accompanying drawings.

### BRIEF DESCRIPTION OF THE SEVERAL VIEWS OF THE DRAWINGS

FIG. 1 is a perspective view of a leading end portion in a surgical device in accordance with an embodiment of the present invention;

FIG. 2 is an exploded perspective view of the leading end portion of the surgical device shown in FIG. 1;

FIG. 3 is a view explaining a wiring state of a wire used in the surgical device in accordance with the present embodiment, and shows a state in which a joint is straight and a state in which the joint is bent;

FIG. 4 is a detailed perspective view of the leading end portion in the surgical device in accordance with the present embodiment;

FIG. 5 is a perspective view showing an entire structure of a manual surgical device in accordance with the embodiment of the present invention;

FIG. 6 is a detailed exploded perspective view of a near-side joint portion in the manual surgical device in accordance with the present embodiment;

FIG. 7 is a view showing a detailed structure of a handle portion in the manual surgical device in accordance with the present embodiment;

FIG. 8 is a view showing a motion aspect of each of portions in the manual surgical device at a time of executing a swing motion on the basis of a near-side operation;

FIG. 9 is a view showing a wiring state of a drive wire between a joint mechanism in a leading end and an operating mechanism in a near side;

FIG. 10 is a perspective view showing an outer appearance of a tension adjusting mechanism of the drive wire and a cross sectional view of the same;

FIG. 11 is a view showing a second structural embodiment of the finger rest executing a gripping motion in the near-side operating mechanism of the surgical device in accordance with the present embodiment;

FIG. 12 is a view showing a force applying portion in the second structural embodiment of the finger rest in accordance with the present embodiment, and a shape change of the finger rest at a time of applying the force;

FIG. 13 is a view showing a third structural embodiment of the finger rest in the surgical device in accordance with the present embodiment;

FIG. 14 is a view showing a detailed structure about the third structural embodiment of the finger rest in accordance with the present embodiment;

FIG. 15 is a view showing an operating state using the third structural embodiment of the finger rest in accordance with the present embodiment and a swing state of the leading end joint portion; and

FIG. 16 is a view showing a used embodiment of the surgical device in accordance with the embodiment of the present invention.

### DETAILED DESCRIPTION OF THE INVENTION

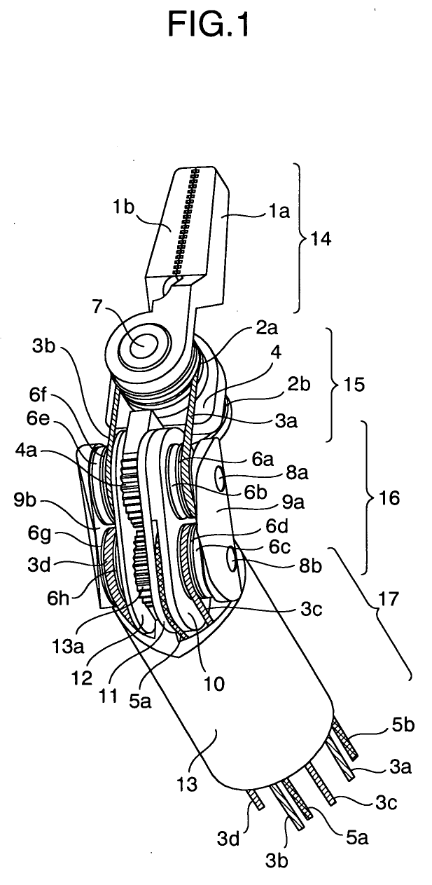

A description will be in detail given below of a surgical device in accordance with an embodiment of the present invention with reference to the accompanying drawings. First, a description will be given of a basic structure of a surgical device in accordance with the embodiment of the present invention with reference to FIGS. 1 to 4.

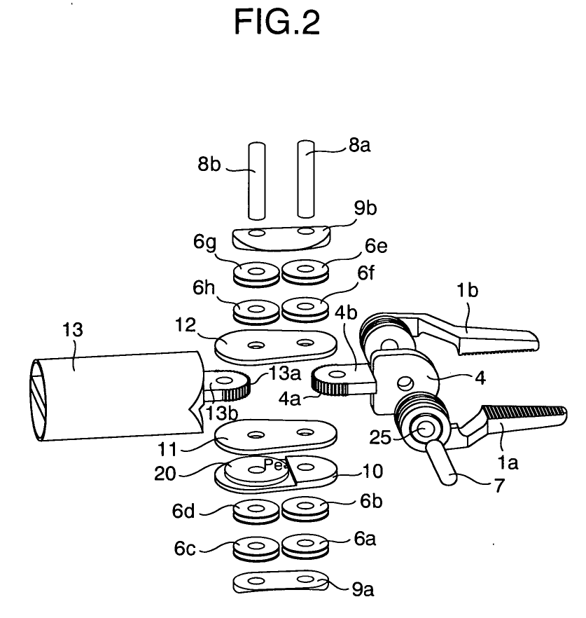

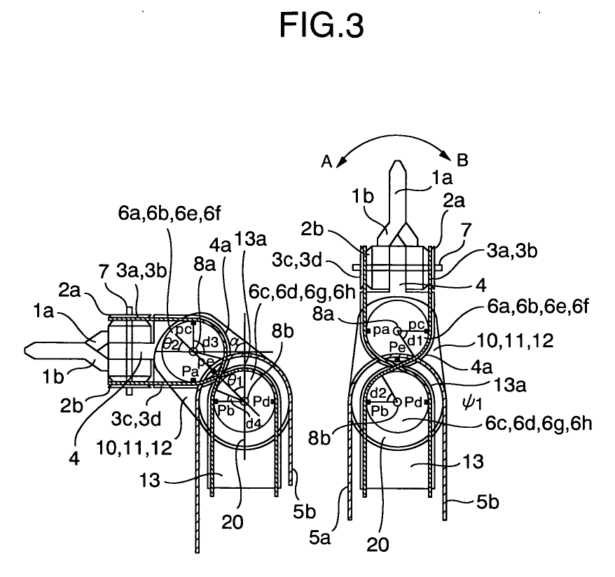

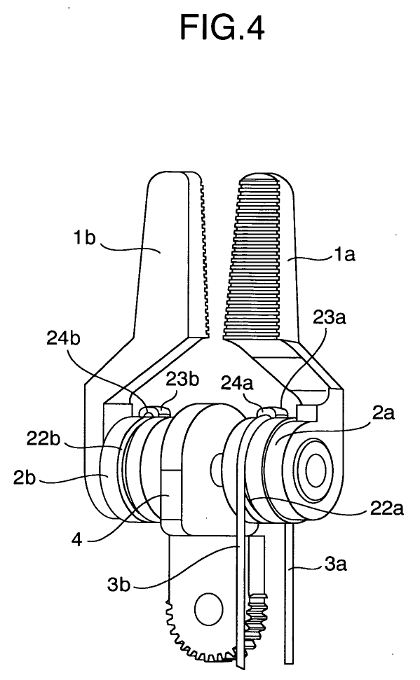

FIG. 1 is a perspective view of a leading end portion in a surgical device in accordance with the present embodiment. FIG. 2 is an exploded perspective view of a leading end portion of the surgical device shown in FIG. 1. In FIG. 2, in order to easily understand, a blade and a wire driving a swing of a joint are omitted. FIG. 3 is a view explaining a wiring state of the wire and shows a state in which the joint is straight and a state in which the joint is bent. FIG. 4 is a detailed perspective view of a leading end portion in the surgical device in accordance with the present embodiment.

In the present embodiment, for a particular description, a description will be given by exemplifying a surgical device for a medical use (the present invention is not particularly limited to the medical surgical device, but can be established as a general structure of an operating device manually operating a grip portion). A leading end portion (hereinafter, refer also to as a leading end joint or an operation device joint) of the surgical device (hereinafter, refer also to as an operation device) is provided with a grip portion (a forceps portion) 14 gripping a suture thread, a suture needle and the like, a leading end portion 15 positioned near a lower portion of the grip portion 14, an intermediate portion 16 forming a second joint together with the leading end portion 15, and a root portion 17 forming a first joint together with the intermediate portion 16, and has drive wires 3a to 3d, 5a and 5b for operating the leading end portion 15 of the grip portion 14 and the intermediate portion 16. The leading end portion serves as a joint of a forceps-shaped operation device. Further, the surgical device is provided with a near-side operation portion (details of which will be mentioned later) which is not illustrated in FIG. 1 and operates a pulling length of the drive wired 3a to 3d, 5a and 5b, in a further near side of the root portion 17, in addition to the leading end portion.

The grip portion 14 has a pair of blades 1a and 1b, and blade pulleys 2a and 2b are arranged in roots of the respective blades 1a and 1b. Grooves 22a and 22b are formed in the blade pulleys 2a and 2b as details of the structure will be shown in FIG. 4. The drive wires 3a to 3d operating the blades are wound around the grooves. The grooves 22a and 22b are provided with holding portions 23a and 23b for holding the wires 3a to 3d in the blade pulleys 2a and 2b.

The leading end portion 15 has a flat plate shaped leading end base portion 4 pinched between a pair of blades 1a and 1b, and a rolling member 4b corresponding to a flat plate approximately orthogonal to the leading end base portion 4 and including a semicircular gear portion 4a (refer to FIG. 2). A hole is formed in a center portion of the leading end base portion 4, and a shaft 7 passes through the hole and a hole formed in a center portion of the blade pulleys 2a and 2b. A hole is formed in a center of the gear portion 4a, and a shaft 8a passing through the hole passes through a hole formed in the intermediate portion 16. In this case, a maximum width of a far side is 5 mm.

The root portion 17 has a cylindrical tube portion 13 and a rolling member 13b positioned at a leading end portion of the tube portion 13 and having a semicircular gear portion 13a formed therein. A hole is formed in a center of the rolling member 13b. In this case, in an illustrated embodiment, the semicircular gear portions 4a and 13a correspond to one means for generating a rolling contact, and include methods such as a process for increasing a friction, a surface treatment of a rubber material, a slip resistance treatment, a connection by a slip resistance wire and the like, in addition to the method using the gear portion.

An intermediate portion 16 is formed between the leading end portion 15 and the root portion 17 in such a manner that the leading end portion 15 and the root portion 17 can rotate around the respective axes of two shafts 8a and 8b. In other words, the intermediate portion 16 has egg-shaped intermediate plates 9b and 12 which are attached to the shafts 8a and 8b and have two holes formed therein, wire guiding pulleys 6e to 6h pinched between the intermediate plates 9b and 12, egg-shaped intermediate plates 11 and 9a which are attached to the shafts 8a and 8b in the same manner and have two holes formed therein, an intermediate plate 10 in which a disc-shaped convex portion 20 is formed in a side through which the shaft 8b passes in adjacent to the intermediate plate 11, and guiding pulleys 6a to 6d pinched between the intermediate plates 10 and 9a (refer to FIG. 2).

The intermediate plate 10 is formed lower around the convex portion 20, and is formed at the same height as the convex portion 20 around the hole through which the shaft 8a passes. A guide path for the wires 5a and 5b are formed by joining the plate 10 formed in the manner mentioned above and the plate 11. The rolling members 4b and 13b are pinched between the plates 11 and 12. The shafts 8a and 8b pass through the hole formed in the plates 9a and 9b, the plates 10 to 12 and the pulleys 6a to 6g. Each of the pulleys 6a to 6g is rotatable around the shafts 8a and 8b, and the rolling members 4b and 13b are rolling contacted in the gear portions 4a and 13a. These members prevent rust or the like from being generated, and are preferably made of a titanium alloy having a light weight and a high rigidity or the like.

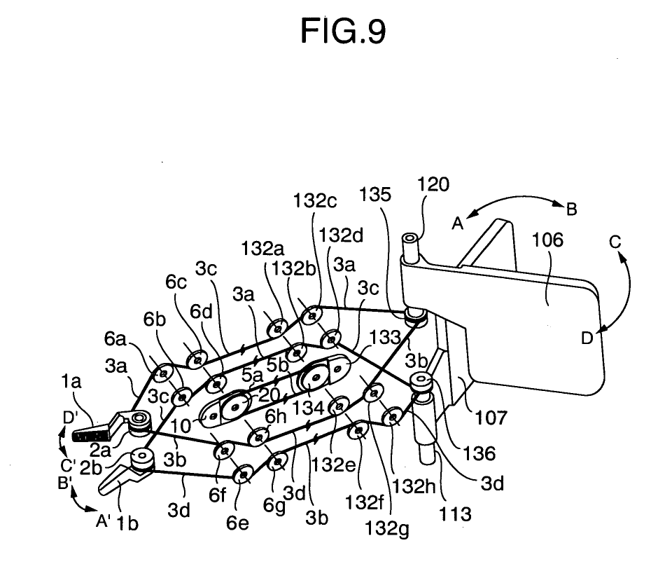

The wires 5a and 5b are fixed to an intersecting point Pe (refer to FIG. 2) with respect to a line connecting centers of two shafts 8a and 8b on a periphery of the convex portion 20 of the intermediate plate 10, and is installed in an outer periphery of the convex portion 20. The wires 5a and 5b in the leading end portion of the surgical device are wound around the near-side joint in the operation device near side as shown in FIG. 9 mentioned below through the inner side of the tube portion 13. The wire 5a and the wire 5b may be constituted by one continuous wire or may be constituted by two wires.

As shown in FIG. 4, grooves 22a and 22b installing the wire thereon are provided in the blade pulleys 2a and 2b. Wire fixing portions 23a and 23b are attached to a part of an outer periphery of the pulleys 2a and 2b. A part of the wires 3a and 3b is fixed in the fixing portions 23a and 23b in accordance with an adhesion, a welding, a brazing, a caulking or the like.

The wire 3a fixed to the blade pulley 2a at one point is fixed to one point on an outer periphery of a finger rest pulley 135 through near-side pulleys 132a and 132c as shown in FIG. 9 mentioned below, after being introduced to the pulley 6a and then to the pulley 6c. In the same manner, the wire 3b fixed to the pulley 2a at one point is introduced to the pulley 6f and then to the pulley 6h, and is fixed to one point on the outer periphery of the finger rest pulley 135. In the present embodiment, the wire 3a and the wire 3b are formed as one continuous wire, however, may be formed as two wires fixed to the blade pulleys 2a and 2b.

The wires 3c and 3d are installed in the blade 1b side, in the same manner as that in the blade 1a side. In other words, the wire 3c fixed to the blade pulley 2b at one point is introduced to the pulley 6b and then to the pulley 6d. At this time, the wire 3c comes across the portion between the pulleys 6b and 6d form a direction intersecting the wire 3a, however, since the heights of the pulleys in which the wire 3a and the wire 3c are installed are differentiated, the wires are not in contact with each other.

The wire 3c is fixed at one point on an outer periphery of a finger rest pulley 136 while passing through near-side pulleys 132b and 132d from the pulleys 6b and 6d. The wire 3d fixed to the blade pulley 2b at one point is introduced to pulleys 6e and 6g in the same manner, and is fixed at one point on the outer periphery of the near-side finger rest pulley 136. Since the height positions of the pulleys 6e and 6f and the pulleys 6g and 6h are changed, the wire 3d is not in contact with the wire 3b.

A description will be given below of an operation of the leading end portion (the leading end joint or the operation device joint) of the surgical device in accordance with the present embodiment provided with the structures mentioned above. The grip portion 14 rotates around the shaft 7 with respect to the leading end portion 15. At this time, if the rotational directions of the shaft 7 and the blades 1a and 1b are the same, the direction of the grip portion 14 is changed, and if the blades 1a and 1b rotate in a reverse direction to the shaft 7, the grip portion 14 executes an opening and closing operation. In particular, when driving the first blade drive source so as to rotate the pulley and pull the wire 3b, the blade 1a moves in a closing direction. On the contrary, when pulling the wire 3a, the blade 1a moves in an opening direction. When driving the second blade drive source so as to rotate the pulley and pull the wire 3c, the blade 1b is closed, and when pulling the wire 3d, the blade 1b is opened. When pulling the wire 3a and the wire 3c together, or pulling the wire 3b and the wire 3d together, the grip portion 14 rotates around the shaft 7 and the grip direction is changed. This is called as a swing motion of the grip portion joint.

As shown in FIG. 3, a swing angle α of the leading end portion 15 is expressed by a sum of an angle θ1 formed by the root portion 17 and the intermediate portion 16, and an angle θ2 formed by the intermediate portion 16 and the leading end portion 15. When operating the near-side operation portion of the surgical device and rotating a plate 133 shown in FIG. 9 mentioned below so as to pull the wire 5a, the intermediate plate 10 is rotated around the shaft 8b in a direction of an arrow A in FIG. 3. At the same time, the shaft 8a, the leading end portion 4, the pulleys 6a, 6b, 6e and 6f and the intermediate plates 9a and 9b are rotated around the shaft 8b in the direction of the arrow A in FIG. 3. At this time, the gear portions 4a and 13a execute a meshing operation while being in rolling contact with each other.

In the case that the gear portion 4a and the gear potion 13a are constituted by the gears having the same dimension, if the intermediate plate 10 is rotated at the angle θ1 around the shaft 8b, the leading end portion 15 is rotated at the angle θ1 around the shaft 8b and is rotated at the angle θ2=θ1 around the shaft 8a therewith. Accordingly, the angle α at which the leading end portion 15 swings with respect to the root portion 17 becomes twice the angle at which the intermediate plate 10 rotates around the shaft 8b. When operating the near-side operation portion of the surgical device, and rotating the plate 133 shown in FIG. 9 mentioned below so as to pull the wire 5b, the leading end portion 14 swings in a direction B in FIG. 3. At this time, in the same manner as the swing in the direction A, the angle α at which the leading end portion 15 swings with respect to the root portion 17 becomes twice the angle at which the intermediate plate 10 rotates around the shaft 8b.

In the case that the radius of the gear portion 4a is R times the radius of the gear portion 13a, if the intermediate plate 10 is rotated at the angle θ1 around the shaft 8b, the leading end portion 15 is rotated at the angle θ2=θ1/R around the shaft 8a. Accordingly, the leading end portion 15 swings at the angle α=θ1(1+1/R) with respect to the root portion 17.

A center angle of the pulley in the portions in which the wires 3a and 3b are in contact with the respective pulleys is changed in accordance with the swing angle α. For example, the wire 3a is in contact with two pulleys 6a and 6c. A sum of the center angles in the portion in which wire 3a is in contact with two pulleys 6a and 6c is expressed by d1+d2 in FIG. 3(a), and d3+d4 in FIG. 3(b). Since the gear portion 4a and the gear portion 13a are engaged and in contact with each other, the value is always fixed, and is not changed in accordance with the swing angle α of the leading end portion joint formed by the root portion 17 and the leading end portion 15. Accordingly, a length of a wire path between the point Pa and the point Pd and a length of a wire path between the point Pc and the point Pb are not changed in accordance with the angle α, and the phase of the wire is not changed.

In this case, a phase of the wire corresponds to an opening and closing angle of the blades 1a and 1b, however, also corresponds to a position of the wire in correspondence to the opening and closing angle, that is, an amount at which the drive portion pulls the wire. Since the phase of the wire is not changed, the blades 1a and 1b are not opened and closed even if the joint of the leading end portion 15 is moved. Accordingly, even if the angle α of the joint of the leading end portion 15 is changed, the length of the path, the phase and the tension of the wire controlling the blades 1a and 1b provided in front of the joint are not affected. As a result, it is possible to transmit only the force applied to the movable portion through the wire as the change of the tension to the hand of the operator operating the operation device joint (the leading end joint).

In accordance with the present embodiment, since the opening and closing angle of the blades 1a and 1b is held constant by operating only the wires 5a and 5b at a time of the swing motion, it is not necessary to regulate the pulling length of the wires 3a to 3d. Further, since the length of the wire path is not changed even if the swing angle is changed, it is possible to prevent a matter that the wire is pulled and the swing angle can not be changed. Further, there is not generated a matter that the wire is loosened at a time when the swing angle is changed.

In accordance with the present embodiment, since there is no interference of the wire, the wire operation can be precisely expressed as the motion of the operation device joint (the leading end joint), and since an operation sensitivity is not changed due to the change of the tension caused by the operation, it is possible to always operate by the same operation feeling. Further, since the force generated in the operation device portion can be transmitted as the change of the tension to the operator, it is possible to feel a condition of a medical treatment operation through a sense of force, and it is possible to operate while feeling such a sense of force as the medical treatment is directly executed by his or her hand as usual. Accordingly, in the case of gripping and sewing a blood vessel and a tissue in the clinical field or the like, it is possible to grip the subject by a suitable force. Accordingly, a minimum invasive and delicate medical treatment can be executed.

Since the swing wire and the grip portion opening and closing wire are independently operated, the motion of the operation device joint becomes stable as well as the operation is easily executed. It is possible to precisely execute the opening and closing motion of the blade at an every swing angle within the movable range and the swing motion while gripping the subject by the blades, and the operator can operate by such an operation feeling as the operator grips the subject by his or her finger without necessity of any complicated operation. Further, since the intermediate portion 16 having two rotation centers is provided, it is possible to enlarge a swing range of the leading end portion 15 with respect to the root portion 17. Accordingly, it is possible to treat affected areas hiding behind an internal organ.

The structure of the grip portion 14, the leading end portion 15, the intermediate portion 16 and the root portion 17 in the present embodiment described above correspond to a structural embodiment employed for a manual surgical device in accordance with the present invention. In the manual surgical device described below, the structure shown in FIGS. 1 to 4 will be employed.

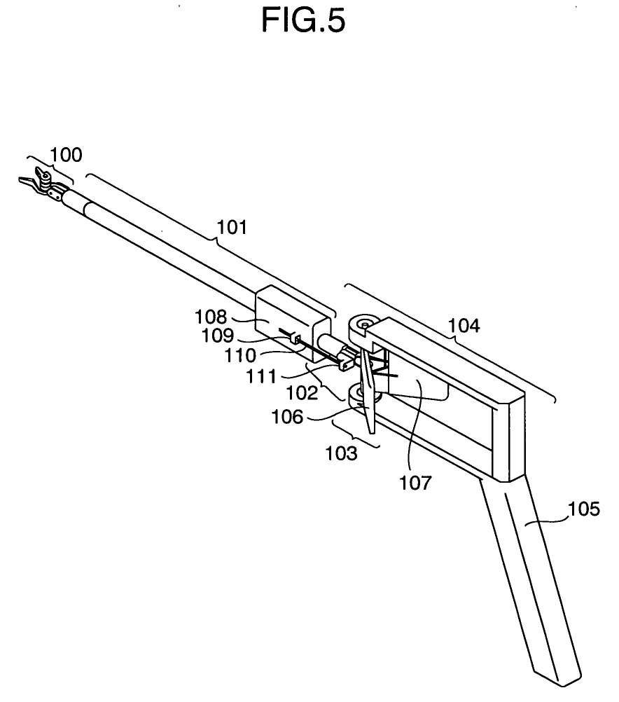

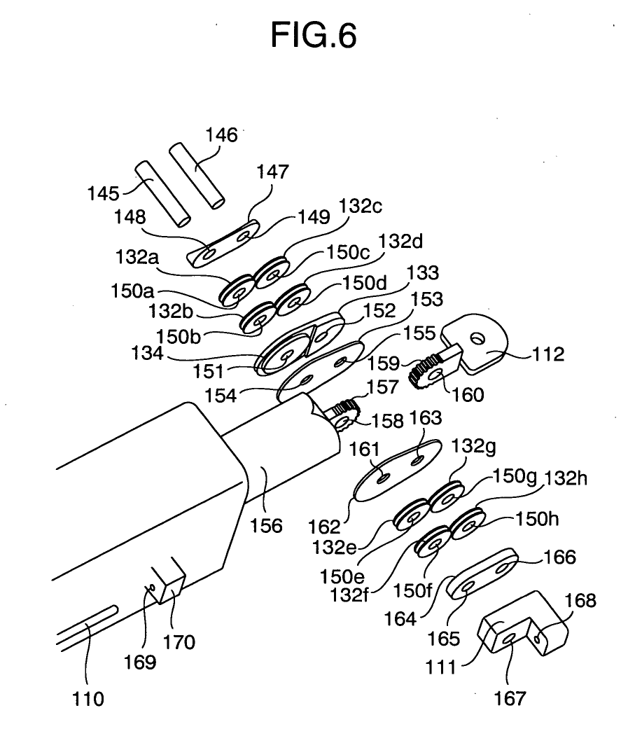

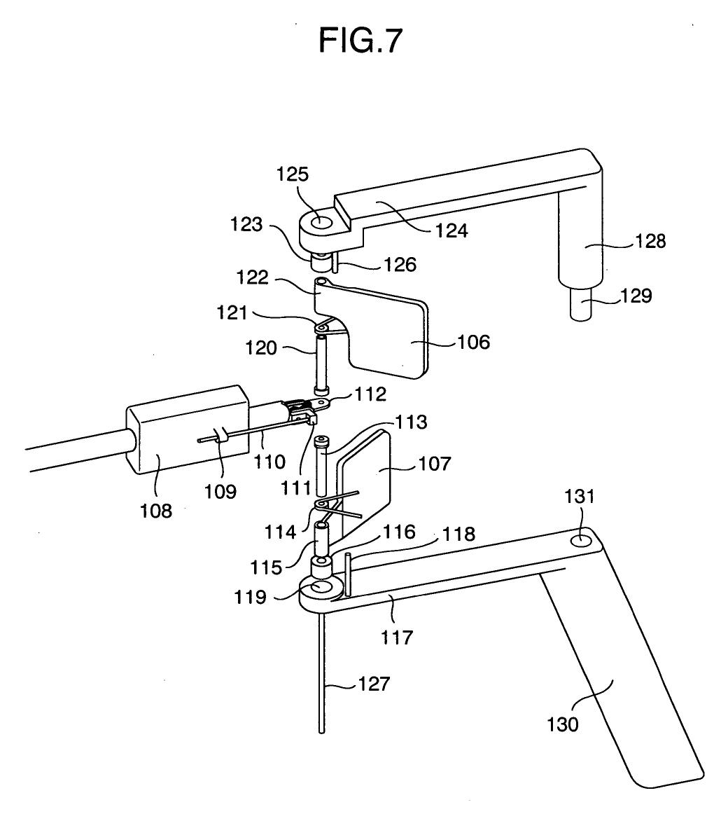

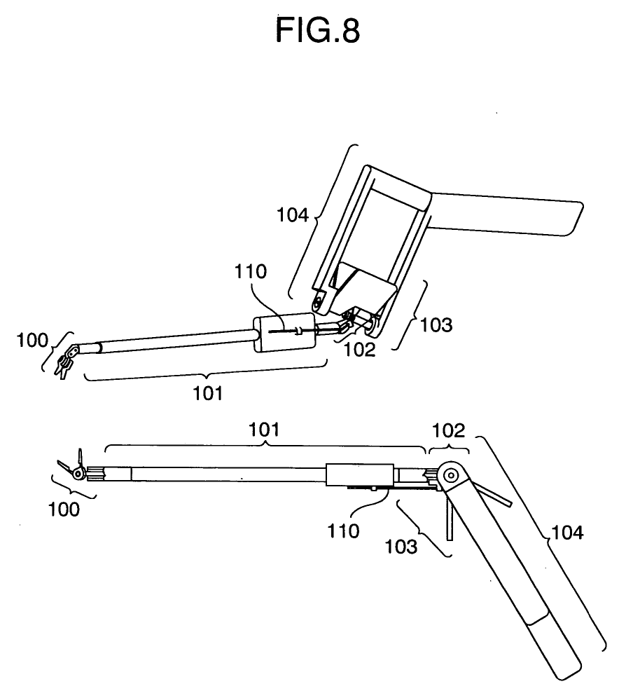

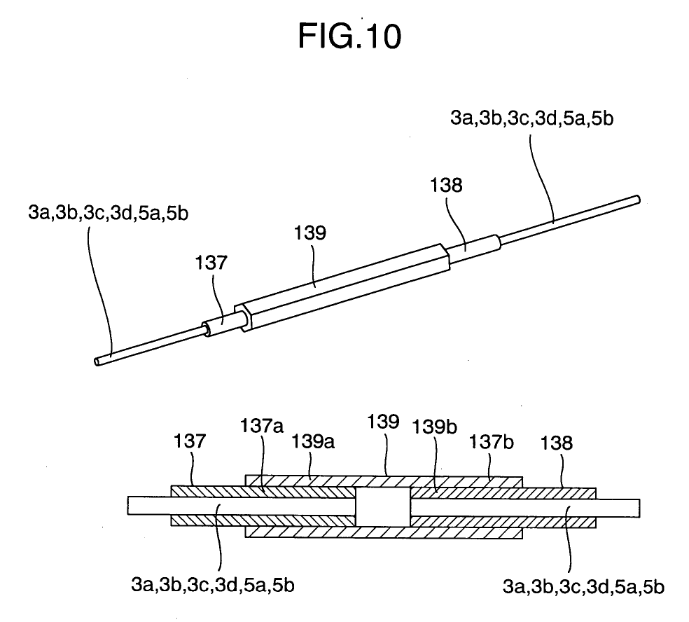

Next, a description will be in detail given below of features of a surgical device in accordance with an embodiment of the present invention with reference to FIGS. 5 to 10. FIG. 5 is a perspective view showing an entire structure of a manual surgical device in accordance with the embodiment of the present invention. FIG. 6 is a detailed exploded perspective view of a near-side joint portion in the manual surgical device in accordance with the present embodiment. FIG. 7 is a view showing a structure of a handle portion in the manual surgical device in accordance with the present embodiment. FIG. 8 is a view showing a motion aspect of each of portions in the manual surgical device at a time of executing a swing motion on the basis of a near-side operation. FIG. 9 is a view showing a wiring state of a drive wire between a joint mechanism in a leading end and an operating mechanism in a near side. FIG. 10 is a perspective view showing an outer appearance of a tension adjusting mechanism of the drive wire and a cross sectional view of the same.

In FIGS. 5 to 10, reference numeral 100 denotes a leading end joint portion, reference numeral 101 denotes an arm portion, reference numeral 102 denotes a near-side joint portion, reference numeral 103 denotes an operating portion, reference numeral 104 denote a handle portion, reference numeral 15 denotes a grip portion, reference numerals 106 and 107 denote a finger rest (one means for opening and closing the blade 1), reference numeral 108 denotes a tension adjusting mechanism, reference numeral 109 denotes an elastic body fixing portion, reference numeral 110 denotes an elastic body, reference numeral 111 denotes a near-side elastic body fixing portion, reference numeral 112 denotes a near-side joint part pulley connecting position, reference numerals 113 and 120 denote an operating pulley, reference numerals 114 and 121 denote an operating finger rest spring, reference numerals 115 and 122 denote a finger rest rotating shaft, reference numerals 116 and 123 denote a bearing, reference numerals 117 and 124 denote a handle beam portion, reference numerals 118 and 126 denote a spring stopper, reference numerals 119 and 125 denote a bearing insertion portion, reference numeral 127 denotes a rotating shaft, reference numeral 128 denotes a handle beam portion coupling portion, reference numerals 129 and 134 denote a convex portion, reference numeral 130 denotes an operator grip portion, reference numeral 131 denotes a concave portion, reference numerals 132a to 132h denote a near-side wire guiding pulley, reference numeral 133 denotes a near-side intermediate plate, reference numerals 135 and 136 denote a finger rest pulley, reference numeral 137 and 138 denotes a wire fixing screw, reference numerals 137a and 138a denote a thread portion, reference numeral 139 denotes a both thread coupling portion, reference numeral 139a denotes a right thread portion, reference numeral 139b denotes a left thread portion, reference numeral 140 denotes a leaf spring type finger rest, reference numeral 141 denotes a rotating shaft portion, reference numeral 142 denotes an operating pulley insertion portion, reference numeral 143 denotes a rotating shaft portion, and reference numeral 144 denotes an operating pulley insertion portion, respectively. In this case, two plate-shaped finger rests 105 and 107 energized with each other in a rotation releasing direction as shown in FIGS. 5 and 7 to 9 correspond to a first structural embodiment of the finger rest.

In FIG. 5, a manual surgical device (hereinafter, refer to as an operation device) in accordance with the embodiment of the present invention is provided with the leading end joint portion 100 shown in FIGS. 1 to 4, the arm portion 101 having the drive wire and the tension adjusting mechanism, the near-side joint portion 102 having the same mechanism as the leading end joint portion 100 and executing a similar operation, the operating portion 103 operated by the operator, the handle portion 104 holding the operating portion 103 and treating the operating portion 103, and the grip portion extended to the handle portion 104 and gripped by the operator with a finger and a palm.

In FIG. 5, the leading end joint portion 100 indicates a combined position of the joint portion and the grip portion in the leading end side, and on the contrary, the near-side joint portion 102 indicates the near side joint portion. The arm portion 101 corresponds to a portion coupling the leading end joint portion 100 and the near-side joint portion 102 and includes the tension adjusting mechanism 108. The operating portion 103 indicates a portion which is relevant to the finger rests 106 and 107. The handle portion 104 indicates a portion which is relevant to the grip (which does not include the finger rests 106 and 107), and is rotatably connected to the finger rest. Further, the operation portion 103 corresponds to a similar structure to the grip portion provided in the joint portion of the leading end joint portion 100, and is connected to the near-side joint portion 102. A function of the operating portion 103 is similar to the leading end joint portion 100, however, a shape thereof is a little different, and the shape and the structure are made taking the operation by the finger into consideration. The operating portion 103 is provided with a torsion spring so as to be open to a predetermined angle (mentioned below). An angle of the operating portion 103 and an opening and closing angle of the grip portion of the leading end joint portion 100 are connected by the drive wire, and execute the opening and closing motion in an interlocking manner.

When operating the handle portion 104 in a direction orthogonal to the opening and closing direction of the operating portion 103, and changing the angle of the near-side joint portion 102 so as to change the angle with respect to the arm portion 101, the angle which the leading end joint portion 100 forms with respect to the arm portion 101 is changed (described in detail in the description of FIG. 8).

Making specific mention of the above matter, the operator executes the opening and closing motion of the blade 1 of the operation device leading end by operating the finger rests 106 and 107 (described later but corresponding to the blade 1 shown in FIG. 1) energized by spring in the opening direction by a thumb, a first finger or a middle finger. Further, when the operator moves the grip portion 105 rightward and leftward and/or upward and downward around the rotational support axis of the finger rests 106 and 107 or the portion near the rotational support axis corresponding to the supporting point, the blade 1 is moved rightward and leftward and/or upward and downward, that is, is swung in a dummy matter with respect to the motion of the operator, on the basis of the relevant motions of the near-side joint portion 102, the drive wire and the leading end joint portion. At this time, the finger rest 106 and 107 can simultaneously shift while keeping a predetermined opening degree. Further, there is provided a tension adjusting mechanism 108 (a detailed structure thereof will be shown in FIG. 10) for adjusting a tension of the drive wire connecting the leading end joint portion 100 and the near-side joint portion 102.

As mentioned above, the blade 1 executes the swing motion on the basis of the operation of the lateral motion and/or the vertical motion of the grip portion 105 integrally structured with the handle portion 104 (which is coupled to the operating portion and is interposed between the operating portion 103 and the grip portion 105), however, since the grip portion 105 is gripped by a palm of the hand at this time, a stability of the operation is improved. Further, since the grip portion 105 can be gripped by the palm of the hand and can be opened and closed by the thumb and the first finger, an operability of the operation device is improved. Further, since the operating portion, the handle portion and the grip portion can be arranged such as to open and close the thumb and the first finger in an unforced natural finger attitude at a time of gripping the grip portion (an installing angle of the grip portion with respect to the handle portion can be set to an appropriate angle more than 90 degree), it is possible to have an effect on an operability, a stability and a fatigue reduction such as the operation of the operation device can be executed in an untiring manner. As a result, it is possible to expect an improvement of a safety of the operation and an improvement of the precision of the medical treatment.

In FIG. 6, a gear portion 157 having a through hole 158 is provided in a tube portion 156 of the near-side joint portion 102, and the gear portion 157 is combined with a gear portion 159 arranged so as to face to the gear portion 157 on the basis of the following particular structure in such a manner as to achieve a rolling contact.

A coupling shaft 145 is inserted to respective through holes 148, 150a, 150b, 151, 154, 158, 161, 150e, 150f and 165 of the intermediate plate 147, the near-side wire guiding pulleys 132a and 132b, the near-side intermediate plates 133 and 153, the gear portion 157, the shaft hole 161, the near-side wire guiding pulleys 132e and 132f, and the intermediate plate 164, thereby coupling the parts. The intermediate plate 153 freely rotates with respect to the gear portion 157 via the coupling shaft 145, and the intermediate plate 133 and the intermediate plate 153 are integrally fixed. The near-side wire guiding pulleys 132a and 132b freely rotate with respect to the near-side intermediate plate 133 and the intermediate plate 147 via the coupling shaft 145. Further, the intermediate plate 162 freely rotates with respect to the gear portion 157 via the coupling shaft 145, and the near-side wire guiding pulleys 132e and 132f freely rotate with respect to the intermediate plate 162 and the intermediate plate 164 via the coupling shaft 145.

In the same manner, a coupling shaft 146 is inserted to respective through holes 149, 150c, 150d, 152, 155, 160, 163, 150g, 150h and 166 of the intermediate plate 147, the near-side wire guiding pulleys 132c and 132d, the near-side intermediate plates 133 and 153, the gear portion 159, the shaft hole 162, the near-side wire guiding pulleys 132g and 132h, and the intermediate plate 164, thereby coupling the parts. Further, the intermediate plate 153 freely rotates with respect to the gear portion 159 via the coupling shaft 146, and the near-side wire guiding pulleys 132c and 132d freely rotate with respect to the intermediate plate 133 and the intermediate plate 147 via the coupling shaft 146.

The intermediate plate 162 freely rotates with respect to the gear portion 159 via the coupling shaft 146, and the near-side wire guiding pulleys 132g and 132h freely rotate with respect to the intermediate plate 162 and the intermediate plate 164 via the coupling shaft 146. An interval between the shaft hole 148 and the shaft hole 149, an interval between the shaft hole 151 and the shaft hole 152, an interval between the shaft hole 154 and the shaft hole 155, an interval between the shaft hole 161 and the shaft hole 163, and an interval between the shaft hole 165 and the shaft hole 166 correspond to a distance at which the gear portion 157 and the gear portion 159 are in rolling contact, and are equal to a total value of a radius of the gear portion 157 (a radius of a pitch circle of the gear) and a radius of the gear portion 159 (a radius of a pitch circle of the gear). A radius of each of the near-side wire guiding pulley 132a to the near-side wire guiding pulley 132h is equal to or less than a half of the total value of the radius of the gear portion 157 (the radius of the pitch circle of the gear) and the radius of the gear portion 159 (the radius of the pitch circle of the gear).

Further, the near-side elastic body fixing portion 111 is provided with a fixing hole 167 and another hole which is invisible in FIG. 6, at the same distance of the two shaft holes 165 and 166 of the intermediate plate 164. The coupling shafts 145 and 146 passing through the element 164 are inserted to two holes provided in the near-side elastic body fixing portion 111 so as to be fixed. Further, the near-side elastic body fixing portion 111 is provided with an elastic body fixing hole 168 in a direction orthogonal to the fixing hole 167, and a diameter of the elastic body fixing hole 168 is set such that the elastic body 110 can be inserted. The elastic body 110 passes through an elastic body insertion hole 169 provided in an elastic body guide portion 170, and is inserted to the elastic body fixing hole 168 of the near-side elastic body fixing portion 111 so as to be fixed.

In FIG. 7, the rotating shaft 127 is inserted to the shaft hole 112 of the near-side joint part pulley connection position 112. The near-side joint part pulley connection position 112 and the rotating shaft 127 may freely rotate or may be fixed therebetween. The operating pulleys 120 and 113 are formed in a hollow shape, and the rotating shaft 127 is rotatably inserted to the hollow portion. As a result, the operating pulleys 120 and 113 are coupled via the rotating shaft 127 so as to be rotatable with the near-side joint part pulley connection position 112.

Further, a ring portion of the operating finger rest spring 121 is rotatably coupled to the operating pulley 120. The operating pulley 120 is inserted to the hollow portion of the finger rest rotating shaft 122 provided in the finger rest 106, and is fixed so as to prevent from being slip with each other. The fixing can be achieved, for example, by using a set screw or the like. At this time, the operating pulley 120 and the finger rest 106 are rotatable with each other before they are fixed by applying a force by the set screw, and can be fixed by screwing the set screw after adjusting a relative angle of rotation thereof. After fixing, when rotating the finger rest 106 around the rotating shaft 127, the operating pulley 120 rotates around the rotating shaft 127 in synchronization with the finger rest 106. The operating pulley 120 is fitted to the bearing 123. Since the bearing 123 is constituted by a bearing, an inner ring is coupled to the operating pulley 120 without any slip. The bearing 123 is inserted to the bearing insertion portion 125 provided in the handle beam portion 124. An outer ring of the bearing 123 and the handle beam portion 124 are coupled without any slip. The inner ring and the outer ring of the bearing 123 are rotatable.

Further, the ring portion of the operating finger rest spring 114 is rotatably coupled to the operating pulley 113. The operating pulley 113 is inserted to the hollow portion of the finger rest rotating shaft 115 provided in the finger rest 107, and is fixed so as not to slip with each other. The fixing can be achieved by using the set screw or the like, in the same manner as mentioned above. At this time, the finger rest rotating shaft 115 and the finger rest 107 are rotatable with each other before they are fixed by applying a force by the set screw, and can be fixed by screwing the set screw after adjusting a relative angle of rotation thereof. After fixing, when rotating the finger rest 107 around the rotating shaft 127, the operating pulley 113 rotates around the rotating shaft 127 in synchronization with the finger rest 107. The operating pulley 113 is fitted to the bearing 116. Since the bearing 116 is constituted by a bearing, an inner ring thereof is coupled to the operating pulley 113 without any slip. The bearing 116 is inserted to the bearing insertion portion 119 provided in the handle beam portion 117. An outer ring of the bearing 116 and the handle beam portion 117 are coupled without any slip. The inner ring and the outer ring of the bearing 116 are rotatable.

The elastic body fixing portion 109 fixing the elastic body 110 is formed in a convex shape from the tension adjusting mechanism 108, and is provided with a through hole to which the elastic body 110 can be inserted and which can move in an inserting direction. The near-side elastic body fixing portion 111 is fixed to the near-side joint, and is provided with a hole to which the elastic body 110 is inserted, and the elastic body 110 and the near-side elastic body fixing portion 111 are fixed (refer to FIG. 6). Since an end portion of the elastic body 110 is fixed to the fixing portions 109 and 111, the elastic body 110 is expanded or compressed by a swing motion of the near-side joint portion 102, generates a force in a direction returning to an original stable state, and achieves a function of preventing the near-side joint portion 102 from being in an unstable state and hanging. In other words, the elastic body 110 holds an original stable position (a reference position for swing) of the near-side joint portion.

One of spring ends of the operating finger rest spring 121 is brought into contact with the spring stopper 126, and an operating range to one side is limited. Another spring end is brought into contact with the finger rest 106. Since one of the spring ends is limited in the operating range by the spring stopper 126, an angle formed by the spring ends becomes small in the case of moving the finger rest 106 around the rotating shaft 127, whereby a force is generated in a direction in which the angle is expanded. Accordingly, a force pushing back the finger rest 106 is generated. Further, in the same manner, one of the spring ends of the operating finger rest spring 114 is brought into contact with the spring stopper 118, and an operating range to one side is limited. Another spring end is brought into contact with the finger rest 107. Since one of the spring ends is limited in the operating range by the spring stopper 118, an angle formed by the spring ends becomes small in the case of moving the finger rest 107 around the rotating shaft 127, whereby a force is generated in a direction in which the angle is expanded. Accordingly, a force pushing back the finger rest 107 is generated.

The spring stopper 126 is provided within the operating range of the finger rest 107 so as to have such a position and a length as not to prevent the operation thereof, and the spring stopper 118 is provided within the operating range of the finger rest 106 so as to have such a position and a length as not to prevent the operation thereof. The handle beam portion 124 is provided with the handle beam portion coupling portion 128 coupled to the handle beam portion 117, the coupling portion 128 is provided with the convex portion 129 achieving an engagement with the concave portion 131, and the handle beam portion 117 is provided with the concave portion 131 achieving the engagement with the convex portion 129. The operator grip portion 130 corresponds to a portion which the operator grips, and is structured such as to have a larger angle than a right angle with respect to the handle beam portion 117, whereby an operability is improved.

FIG. 8 shows a near-side operating state and a swing state of the joint in the leading end. In FIG. 8, when the handle portion 104 and the operating portion 103 are turned in a direction orthogonal to the opening and closing direction of the finger rests 106 and 107 of the operating portion 103 by the joint of the near-side joint portion 102, the joint of the leading end joint portion 100 swings in a reverse direction to that of the joint of the near-side joint portion 102 in the leading end, as shown in FIG. 8. Further, as illustrated, when operating an entire of the operating portion 103 in the opening and closing direction of the finger rest of the operating portion 103, the grip portion of the joint of the leading end joint portion 100 swings in the reverse direction to the operating direction of the operating portion 103. At this time, since the finger is set on the finger rests 106 and 107, the handle portion 104 gripped by the operator is changed at a similar angle to that of the operating portion 103. In the example of an upper drawing in FIG. 8, when moving the handle portion 104 and the operating portion 103 upward and downward, the grip portion of the leading end joint portion 100 moves upward and downward. Further, in the example of a lower drawing in FIG. 8, when moving the handle portion 104 and the operating portion 103 rightward and leftward, the grip portion of the leading end joint portion 100 moves rightward and leftward so as to swing.

As shown in FIG. 8, since the direction of the angular change of the joint of the near-side joint portion 102 in the near side becomes reverse to the direction of the angular change of the joint of the leading end joint portion 100 in the leading end, the extending direction of the finger in the near side and the direction of the grip portion of the leading end joint portion 100 are always directed to the same direction. Accordingly, it is possible to operate the grip portion of the operation device in such a feeling that the operator directly operates by his or her finger.

FIG. 9 shows an interconnection of the wire and an operating condition. A description will be given by using the parts shown in FIGS. 2, 6, 7 and 8.

In the near-side intermediate plate 133 and the leading end side intermediate plate 10, the wire is arranged on the loop in such a manner as to be along the convex portion 134 and the convex portion 20 of the respective parts. In the wire loop, the convex portion 134 and the convex portion 20, the drive wires 5a and 5b are operated on the basis of the operation of the convex portion 134 to which each of the wire loop, the convex portion 134 and the convex portion 20 is fixed in at least one position, and the force is transmitted to the leading end side convex portion 20 on the basis of the operation of the drive wires 5a and 5b.

As shown in an upper drawing of FIG. 8, since the operating portion 103 and the handle portion 104 are connected to the near-side joint part pulley connecting portion 112 as shown in FIGS. 6 and 7, when operating the operating portion 103 and the handle portion 104 in a direction orthogonal to the opening and closing direction, the pulley connecting portion 112 executes a rotating motion around the coupling shaft 146 passing through the shaft hole 160. Accordingly, since the gear portion 159 and the gear portion 157 are in the relation of rolling contact as shown in FIG. 6, the near-side joint part pulley connecting position 112 executes a rotating motion around the coupling shaft 145 passing through the shaft hole 158. Then, the wires 5a and 5b rotate the intermediate plate 10 around the shaft 8b in FIG. 2.

As a result, the intermediate plates 9b, 12 and 11 in FIG. 2 execute the rotation around the shaft 8b in the same manner as the intermediate plate 10, so that the gear portion 4a coupled by the intermediate plates 9b, 12, 11 and 10 rotates around the gear portion 13a, and the gear portion 13a and the gear portion 4a are in a rolling contact relation therebetween. Accordingly, the gear portion 4a executes the rotation around the shaft 8a. Therefore, it is possible to bent the joint portion of the leading end joint portion 100 provided in the leading end in the direction orthogonal to the opening and closing direction of the grip portion of the leading end joint portion 100, by operating the operating portion 103 and the handle portion 104 in the direction orthogonal to the opening and closing direction of the operating portion 103.

At this time, since the drive wires 3a, 3b, 3c and 3d are structured, as shown in FIG. 3, such that the length of the path is uniform, there appears no phenomenon that the opening and closing angle of the blades 1a and 1b is changed in the swing motion on the basis of the operation of the drive wires 5a and 5b. Further, since the change of tension is not generated in the drive wires 3a, 3b, 3c and 3d in the swing motion by the drive wires 5a and 5b, the drive wires 3a to 3d generate neither friction nor obstacle for the swing motion by the drive wires 5a and 5b. Accordingly, it is possible to operate by a light force.

The above matters can be applied similarly to the joint portion 102 in the near side, in addition to the joint portion 100 in the leading end. Since the length of the path is not changed in the drive wires 3a to 3b working with the finger rests 106 and 107 at a time of operating the operating portion 103 and the handle portion 104 so as to execute the swing motion via the drive wires 5a and 5b, no force is generated from the drive wire in the direction in which the opening and closing direction of the finger rest arbitrarily changes without the operation of the operator. Further, since the change of tension is not generated in the drive wires 3a to 3b, the operating force required at a time of operating the operating portion 103 and the handle portion 104 can be made small.

In the loop of the wire connected to the blade 1a from the finger rest 106, and the loop of the wire connected to the blade 1b from the finger rest 107, the finger rest and the grip portion work with each other. In this case, a description will be given by exemplifying the wire loop connected to the blade la from the finger rest 106. The finger rest 106 and the operating pulley 120 are fixed. The wires 3a and 3b are connected to the groove-of the finger rest pulley 135 provided in the operating pulley 120. The wires 3a and 3b may be constituted by one wire or independent wires, however, the wires 3a and 3b and the finger rest pulley 135 are fixed at one position, and the wire is pulled working with the rotation of the finger rest pulley 135.

The wire 3a is introduced to the near-side wire guiding pulley 132c, is guided through the portion between the pulley 132c and the other near-side wire guiding pulley 132a and along the wire guiding pulley 132a, and is introduced to the wire guiding pulley 6c. As shown in FIGS. 1 and 3, the wire is introduced to the wire guiding pulley 6 through the portion between the wire guiding pulleys 6c and 6a, and is introduced to the blade pulley 2a from the pulley 6a.

The grip portion (the blade) 1a is fixed to the blade pulley 2a. The wire 3a is fixed to the blade pulley 2a, is introduced as the wire 3b to the wire guiding pulley 6f, passes through the portion between the wire guiding pulleys 6f and 6h, and is introduced to the near-side wire guiding pulley 132e along the pulley 6h. Further, the wire passes through the portion between the near-side wire guiding pulleys 132e and 132g, is introduced to the pulley 132g and is returned to the finger rest pulley 135. The wire 3b is fixed in the finger rest pulley 135, and forms the loop with the wire 3a. Further, the same structure is applied to the loop of the wire connected to the blade 1b from the other finger rest 107.

In this case, when operating the finger rests 106 and 107 in a direction in which they are closed with each other (a direction C and a direction B shown in FIG. 9), the wires 3b and 3c are pulled, and the blades 1a and 1b are operated in a direction in which they are closed with each other (a direction C′ and a direction B′ shown in FIG. 9). Further, when simultaneously operating the finger rests 106 and 107 in the direction in which the finger rest 106 is closed (the direction C and the direction A shown in FIG. 9), the wires 3b and 3d are pulled, and the blades 1a and 1b are all together operated in the direction in which the blade 1a is closed (the direction C′ and the direction A′). Accordingly, it is possible to achieve the swing motion of the entire of the grip portion. In the swing motion to the opposite side, when simultaneously operating the finger rests 106 and 107 in a direction in which the finger rest 107 is closed (a direction D and a direction B), the wires 3a and 3d are pulled, and the blades 1a and 1b are all together operated in a direction in which the blade 1b is closed (a direction D′ and a direction B′). As mentioned above, the swing motion can be achieved as shown in the lower drawing of FIG. 8. At this time, even if the near-side intermediate plate 133 and the intermediate plate 10 are rotated so as to be as shown in the upper drawing of FIG. 8, neither the change of the tension nor the change of the path length are generated in the wires 3a to 3b on the basis of the angle of rotation in the near-side intermediate plate 133 and the intermediate plate 10. Accordingly, it is possible to achieve the opening and closing motion of the grip portion and the swing motion of the grip portion on the basis of the same light force as that in the case that the near-side intermediate plate 133 and the intermediate plate 10 are not angularly changed.

As mentioned above, when operating the swing to two orthogonal directions, the operating force is not changed in correspondence to the respective angles, but it is possible to always operate by the light force. Accordingly, it is possible to execute an open-ended operation flexibly. Since the tension applied to the wire is always constant regardless of the angle of the swing, the space at a time of gripping is transmitted as the tension change of the wire to the near-side finger rests 106 and 107 regardless of the swing angle in the case of gripping something by the blades 1a and 1b. Accordingly, it is possible to feel a sense gripping something and a griping strength by the finger. Further, even when the leading end of the operation device comes into collision with something at a time of swinging, the tension of the wire is changed on the basis of the obstruction of the angular change at that time. Accordingly, the tension change can be felt by a fingertip via the wire connected to the finger rests 106 and 107. Therefore, as a medical manipulator, it is possible to feel the contact with the peripheral internal organ or the like during the operation, and it is possible to increase a safety.

In FIG. 10, the wire fixing screws 137 and 138 are provided in the middle of the wires 3a to 3d and the wires 5a and 5b. The wire fixing screws 137 and 138 have threads formed in outer sides, and are screwed into the both thread coupling portion 139 having a female thread. It is possible to adjust the lengths of the wires 3a to 3d and the wires 5a and 5b on the basis of how much the wire fixing screws 137 and 138 are screwed into the both thread coupling portion 139. The adjusting portion is stored in an inner side of the tension adjusting mechanism 108 shown in FIG. 7. It is possible to adjust the tension by adjusting the length every one of the wires.

It is preferable that the tension is adjusted in accordance with the following procedure. First, the wire fixing screw 137 is fixed to the end portions of the wires 3a to 3d and the wires 5a and 5b extending from the blade pulleys 2a and 2b and the intermediate plate 10. The wire fixing screw 138 is fixed to the end portions of the wires 3a to 3d and the wires 5a and 5b extending from the finger rest pulleys 135 and 136 and the near-side intermediate plate 133. The wire fixing screws 137 and 138 are screwed into the both thread coupling portion 139 in the inner portion of the tension adjusting mechanism 108, and the tension is applied until a slack of each of the wires is lost. The finger rests 106 and 107 are fixed to the operating pulleys 120 and 113 by closing the blades 1a and 1b after the tension is applied, and rotating the finger rests 106 and 107 around the operating pulleys 120 and 113, under an attitude of being just closed or being open at some degrees. The fixing method can be achieved by applying the pressure by the set screw or the like.

In accordance with the above procedures, it is possible to apply the tension of the wire and make the attitude of the blades 1a and 1b in conformity to the attitude of the finger rests 106 and 107. This can be achieved because the operating pulleys 120 and 113 and the finger rests 106 and 107 can be separated, and the position fixing the finger rests 106 and 107 can be adjusted, in addition to the provision of the mechanism for adjusting the tension of the wire.

The above description is given of the particular structure of the surgical device in accordance with the embodiment of the present invention, and the function and the operation thereof. A description will be given next of various structures of the finger rest in the surgical device in accordance with the present embodiment, and the function and the operation thereof. Further, a description will be given of a used aspect of the surgical device in accordance with the present embodiment.

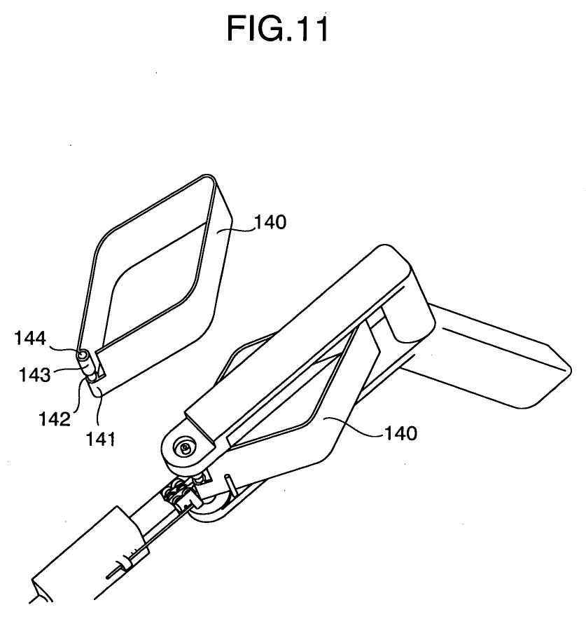

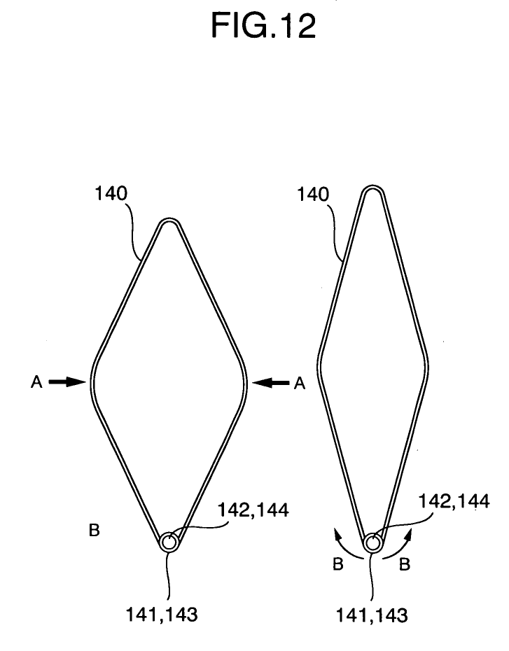

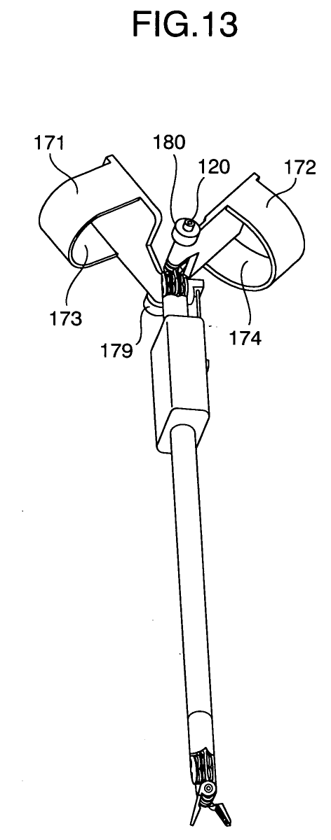

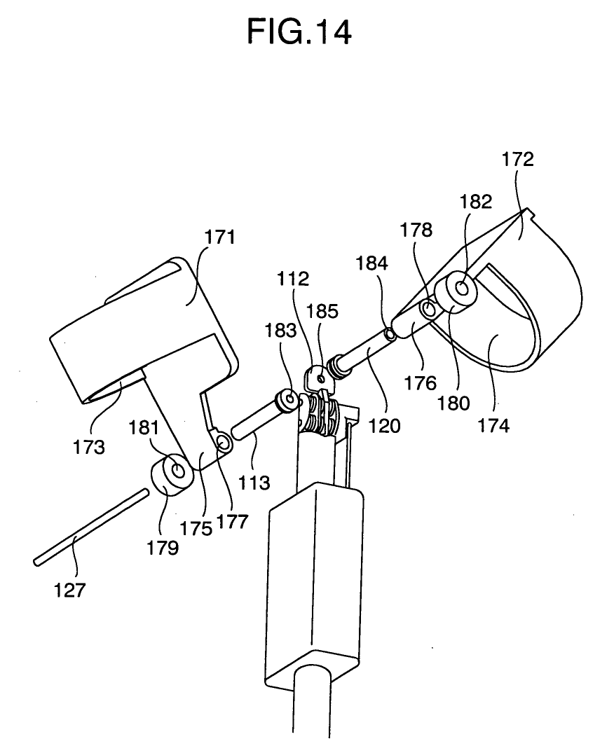

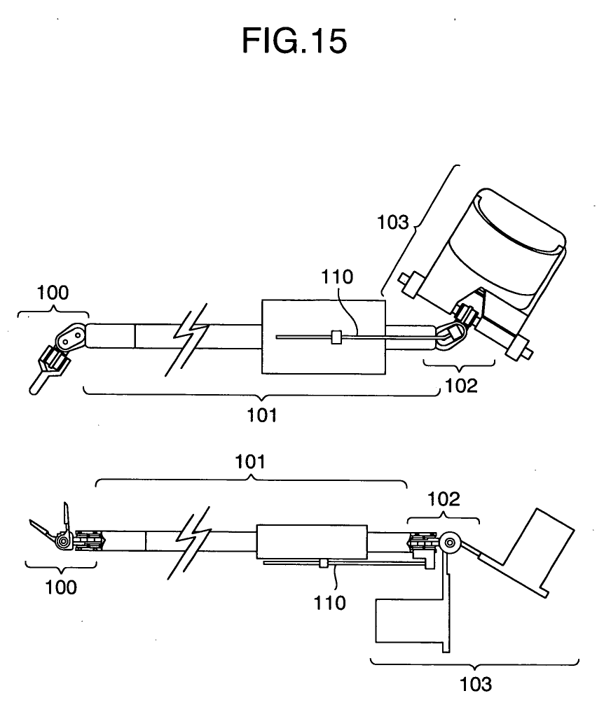

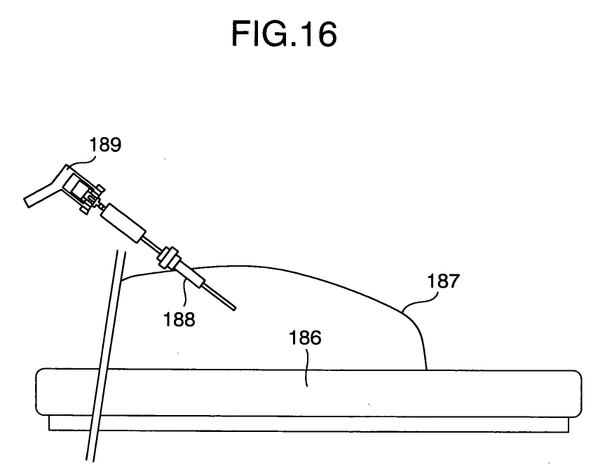

In this case, FIG. 11 is a view showing a second structural embodiment of the finger rest executing the gripping motion in the near-side operating mechanism of the surgical device in accordance with the present embodiment, and FIG. 12 is a view showing a force applying portion in the second structural embodiment of the finger rest in accordance with the present embodiment, and a shape change of the finger rest at a time of applying the force. FIG. 13 is a view showing a third structural embodiment of the finger rest in the surgical device in accordance with the present embodiment, FIG. 14 is a view showing a detailed structure about the third structural embodiment of the finger rest in accordance with the present embodiment, and FIG. 15 is a view showing an operating state using the third structural embodiment of the finger rest in accordance with the present embodiment and a swing state of the leading end joint portion. FIG. 16 is a view showing a used embodiment of the surgical device in accordance with an embodiment of the present invention.

In FIG. 11, there is shown a second structural embodiment of an operating end portion (a finger rest) which is gripped or held by a finger (for example, a thumb, a first finger, a middle finger or the like) by the operator. The second structural embodiment corresponds to the leaf spring type finger rest 140, and the finger rest 140 is structured by a spring-like material. The mounting method is constituted by steps of first expanding the leaf spring type finger rest 140, inserting the operating pulleys 120 and 113 to the operating pulley insertion portions 144 and 142, attaching them to the near-side joint part pulley connecting position 112 and passing through the axis of the rotating shaft 127. In this second structural embodiment, since the finger rest has a spring characteristic, it is not necessary to use a torsion bar.

An operating aspect of the second structural embodiment of the finger rest is shown by using FIG. 12. When pushing a position A by a finger as shown in a left drawing of FIG. 12, the finger rest is deformed as shown in a right drawing. At this time, since the rotating shaft portions 141 and 143 rotate in a direction B in FIG. 12, the operating pulleys 120 and 113 inserted and fixed to the operating pulley insertion portions 142 and 144 also rotate, and it is possible to wind up the wire. Further, when rotating the leaf spring type finger rest 140 itself around the operating pulley inserting portions 142 and 144, it is possible to execute the swing motion changing the direction of the grip portion (the blade) in the leading end of the surgical device.

When loosening the pinching force as illustrated by reference symbol A, the finger rest 140 is returned to the original shape, and the rotation of the operating pulley insertion portions 142 and 144 turns back. Accordingly, the opening and closing angle of the grip portion (the blade) turns back. If the fixing of the rotating shaft portions 141 and 143 and the operating pulleys 120 and 113 is executed after applying the tension to the wire and suitably adjusting the attitude of the leaf spring type finger rest 140 in a state of closing the grip portion of the leading end joint portion 100, it is possible to adjust the relative attitude of the grip portion and the finger rest as well as adjusting the tension of the wire.

In a third structural embodiment of the finger rest shown in FIG. 13, rings 173 and 174 for inserting the finger are provided in finger rests 171 and 172. The rings 173 and 174 may be formed in a ring shape of a rigid body, or may be structured by a raw material having a flexibility. In accordance with the third structural embodiment, since it is possible to execute a motion of actively opening by the finger, it is not necessary to use the torsion spring. Further, since the operation device can be held by inserting the finger to the ring, it is possible to operate the operation device even if the position gripped by the operator's hand such as the handle portion 104 is not provided.

A description will be given of a detailed structure of the third structural embodiment shown in FIG. 14. The rotating shaft 127 is inserted to a shaft hole 185 of the near-side joint part pulley connection position 112, and the rotating shaft 127 is inserted to hollow portions 183 and 184 provided in the operating pulleys 113 and 120. The operating pulleys 113 and 120 are rotatable with respect to the rotating shaft 127.

The operating pulleys 113 and 120 are respectively inserted to hollow portions 177 and 178 provided in rotating shafts 175 and 176 of the finger rests 171 and 172. The finger rest 171 is rotatable with respect to the operating pulley 113, however, can be fixed thereto by a set screw or the like. The finger rest 172 is rotatable with respect to the operating pulley 120, however, can be fixed thereto by a set screw or the like. The end portion of the operating pulley 113 passing through the shaft hole 177 is inserted to the through hole 181 of the fixing ring 179, and the fixing ring 179 is fixed to the operating pulley 113 by the set screw or the like, thereby preventing the finger rest 171 from coming off from the operating pulley 113 even in a state in which the finger rest 171 is not fixed to the operating pulley 113. In the same manner, the end portion of the operating pulley 120 passing through the shaft hole 178 is inserted to the through hole 182 of the fixing ring 180, and the fixing ring 180 is fixed to the operating pulley 120 by the set screw or the like, thereby preventing the finger rest 172 from coming off from the operating pulley 120 even in a state in which the finger rest 172 is not fixed to the operating pulley 120.

A description will be given of an operation aspect in a third structural embodiment of the finger rest and a motion aspect of the leading end joint portion by using FIG. 15. When operating the operating portion 103 in a direction orthogonal to the opening and closing direction, the leading end joint portion 100 swings in the joint portion as shown by an upper drawing of FIG. 15. A swing direction is the same as the case of the structural embodiment 1 in accordance with the present embodiment, and corresponds to a reverse direction to the near-side joint portion 102. At this time, the elastic body 110 is bent on the basis of the swing of the near-side joint portion 102, generates a force in a directly returning direction, and prevents the near-side joint portion 102 from unstably oscillating (swinging). When operating the operating portion 103 in the opening and closing direction, the grip portion of the leading end joint portion 100 swings as shown by a lower drawing of FIG. 15. A swing direction is the same as the case of the structural embodiment 1 in accordance with the present embodiment, and is reverse to the swing direction of the operating portion 103 with respect to the near-side joint portion 102.

Since the swing direction of the joint is reverse between the leading end joint portion 100 and the near-side joint portion 102, it is possible to operate such that the leading end of the joint is always directed to the finger extending direction. Accordingly, it is possible to execute a visceral operation, and it is possible to improve an operability.

FIG. 16 shows an embodiment at a time of practically using the surgical device in accordance with the present embodiment. It is possible to execute the swing motion and the opening and closing motion viscerally and in a dummy manner with respect to the grip portion in the leading end, by inserting a trockar 188 to a dissection portion of a patient 187 on an operation bed 186, inserting an operation device 189 to the trockar 188 and operating the operating portion 103.

A description will be mainly given above of the structure relating to the surgical device in accordance with the embodiments of the present invention. A description will be given of functions, operations and effects of the surgical device provided with the structure mentioned above. In the present embodiment, since the grip portion in the leading end joint portion has the wide movable range and is in multiple degrees of freedom, it is possible to bend the grip portion (the blade) at multiple degrees of freedom even in a comfortable position so as to approach to the affected part in the open-ended attitude. Accordingly, it is not necessary to operate by an entire of the arm. Further, since it is possible to operate in an attitude that an under arm is closed, it is possible to execute a delicate operation, so that the safety is increased.

Further, since the leading end joint portion can be operated in the open-ended attitude on the basis of a motion of a wrist, such a physical fatigue of the operator as to interrupt the medical treatment work is hard to be generated, and it is possible to shorten the time for the medical treatment. Since it is possible to simultaneously execute the operation of the finger rest and the operation of the swing for opening and closing, it is possible to obtain such a sense as to directly execute the medical treatment by his or her hand more viscerally, it is possible to improve an operability and a safety can be improved.

Since the leading end joint portion can move in multiple degrees of freedom in the large swing movable range at the positioned area, a flexible motion sufficiently utilizing the wide movable range can be effectively used for the medical treatment operation in a narrow visual field observed by an endoscope.

Further, since the structure is made such that the degrees of freedom of the joints are not interfered with each other (the structure provided with three wire paths shown in FIG. 9), each of them can have a wide movable range. Since the degree of freedom of the leading end joint portion transmits the driving force through the degree of freedom of the near-side joint portion by the wire, and the length of the wire path is not changed even if the swing is executed on the basis of the degree of freedom of the near-side joint portion, the external force by the swing is not applied to the wire running into the leading end joint portion, and it is not interfered with the other degree of freedom.

Further, the operability can be changed by changing a rate of the diameters of the finger rest pulleys 135 and 136 in the near-side joint portion and the diameters of the blade pulleys 2a and 2b in the leading end joint portion, and a rate of the diameter of the convex portion 134 in the near-side intermediate plate 133 and the diameter of the convex portion 20 of the intermediate plate 10 in the leading end joint portion. In other words, when making the diameter of the pulley and the diameter of the convex portion in the near side larger than the diameter of the pulley and the diameter of the convex portion in the leading end side, it is possible to enlarge the motion of the near side on the basis of the rate so as to transmit to the operation device in the leading end. Accordingly, it is possible to make the movable range of the wrist small, and it is possible to lighten the load of the wrist. On the contrary, in the case of making the diameter of the pulley and the diameter of the convex portion in the near side smaller than the diameter of the pulley and the diameter of the convex portion in the leading end side, it is possible to reduce the motion in the near side on the basis of the rate so as to transmit to the operation device in the leading end. Therefore, it is possible to achieve a finer motion by the operation device in the leading end. Further, since the operating force of the wrist is increased in accordance with the rate, it is possible to execute the medical treatment operation by a lighter force.

Further, it is possible to obtain such an operability as to directly execute the medical treatment operation by his or her finger, by employing the similar shape that the position corresponding to the leading end grip portion (the blade) is set as the finger rest, and it is possible to convert the motion of the wrist directly and in a dummy manner into the motion of the operation device in the leading end.

It should be further understood by those skilled in the art that although the foregoing description has been made on embodiments of the invention, the invention is not limited thereto and various changes and modifications may be made without departing from the spirit of the invention and the scope of the appended claims.

1. A surgical device comprising:
a leading end joint portion having a leading end grip portion;
a near-side joint portion having an operation portion;
a handle portion supporting said operation portion; and
an arm portion storing a wire for linking motions of said leading end joint portion and said near-side joint portion,
wherein said leading end joint portion is moved downward and upward by operating said operation portion and said handle portion upward and downward around said near-side joint portion, and said leading end joint portion is moved rightward and leftward by operating said operation portion and said handle portion leftward and rightward, thereby making said leading end joint portion execute a swing motion, and
wherein said leading end grip portion is opened and closed by opening and closing said operation portion.

2. A surgical device as claimed in claim 1, wherein a pair of rolling contact means achieving a rolling contact are provided in said leading end joint portion and said near-side joint portion,
wherein a pulley is provided in one rolling contact means of said pair of rolling contact means in said leading end joint portion and said near-side joint portion, said leading end grip portion is attached to said pulley in said leading end joint portion and said operation portion is attache to said pulley in said near-side joint portion, and a wire is wound around said pulley of said leading end joint portion and said near-side joint portion,
wherein a wire is wound around the other rolling contact means in said pair of rolling contact means in said leading end joint portion and said near-side joint portion,
said leading end joint portion is swung on the basis of a rotational drive via the wire in the other rolling contact means in said pair of rolling contact means and a rotational drive via the wire in said pulley, by moving said operation portion and said handle portion upward and downward, and rightward and leftward, and
wherein said leading end grip portion is opened and closed on the basis of a rotational drive via the wire in said pulley, by opening and closing said operation portion.

3. A surgical device as claimed in claim 1, wherein said operating portion is provided with two opening and closing means which are energized with each other in a rotation releasing direction.

4. A surgical device as claimed in claim 2, wherein said operating portion is provided with two opening and closing means which are energized with each other in a rotation releasing direction.

5. A surgical device as claimed in claim 1, wherein said operating portion is constituted by a spring material having an approximately rhomboid shape with a cut line in one corner, and is provided with an opening and closing means pinching a bulge portion of said approximately rhomboid shape.

6. A surgical device as claimed in claim 2, wherein said operating portion is constituted by a spring material having an approximately rhomboid shape with a cut line in one corner, and is provided with an opening and closing means pinching a bulge portion of said approximately rhomboid shape.

7. A surgical device as claimed in claim l, wherein said arm portion includes a tension adjusting mechanism adjusting a tension of said wire operating said leading end joint portion and said near-side joint portion in an interlocking manner, and
wherein a stable position of said near-side joint portion is held by connecting an expanding and compressing elastic body to a portion between said tension adjusting mechanism and said near-side joint portion.

8. A surgical device as claimed in claim 2, wherein said arm portion includes a tension adjusting mechanism adjusting a tension of said wire operating said leading end joint portion and said near-side joint portion in an interlocking manner, and
wherein a stable position of said near-side joint portion is held by connecting an expanding and compressing elastic body to a portion between said tension adjusting mechanism and said near-side joint portion.

9. A surgical device as claimed in claim 3, wherein said arm portion includes a tension adjusting mechanism adjusting a tension of said wire operating said leading end joint portion and said near-side joint portion in an interlocking manner, and
wherein a stable position of said near-side joint portion is held by connecting an expanding and compressing elastic body to a portion between said tension adjusting mechanism and said near-side joint portion.

10. A surgical device as claimed in claim 4, wherein said arm portion includes a tension adjusting mechanism adjusting a tension of said wire operating said leading end joint portion and said near-side joint portion in an interlocking manner, and
wherein a stable position of said near-side joint portion is held by connecting an expanding and compressing elastic body to a portion between said tension adjusting mechanism and said near-side joint portion.

11. A surgical device as claimed in claim 5, wherein said arm portion includes a tension adjusting mechanism adjusting a tension of said wire operating said leading end joint portion and said near-side joint portion in an interlocking manner, and
wherein a stable position of said near-side joint portion is held by connecting an expanding and compressing elastic body to a portion between said tension adjusting mechanism and said near-side joint portion.

12. A surgical device as claimed in claim 6, wherein said arm portion includes a tension adjusting mechanism adjusting a tension of said wire operating said leading end joint portion and said near-side joint portion in an interlocking manner, and
wherein a stable position of said near-side joint portion is held by connecting an expanding and compressing elastic body to a portion between said tension adjusting mechanism and said near-side joint portion.

13. A surgical device as claimed in claim 1, wherein a grip portion held by a palm is provided in an extending manner in said handle portion.

14. A surgical device as claimed in claim 2, wherein a grip portion held by a palm is provided in an extending manner in said handle portion.

15. A surgical device as claimed in claim 3, wherein a grip portion held by a palm is provided in an extending manner in said handle portion.

16. A surgical device as claimed in claim 4, wherein a grip portion held by a palm is provided in an extending manner in said handle portion.

17. A surgical device as claimed in claim 5, wherein a grip portion held by a palm is provided in an extending manner in said handle portion.

18. A surgical device as claimed in claim 6, wherein a grip portion held by a palm is provided in an extending manner in said handle portion.

19. A surgical device as claimed in claim 7, wherein a grip portion held by a palm is provided in an extending manner in said handle portion.

20. A surgical device as claimed in claim 8, wherein a grip portion held by a palm is provided in an extending manner in said handle portion.

21. A surgical device as claimed in claim 9, wherein a grip portion held by a palm is provided in an extending manner in said handle portion.

22. A surgical device as claimed in claim 10, wherein a grip portion held by a palm is provided in an extending manner in said handle portion.

23. A surgical device as claimed in claim 11, wherein a grip portion held by a palm is provided in an extending manner in said handle portion.

24. A surgical device as claimed in claim 12, wherein a grip portion held by a palm is provided in an extending manner in said handle portion.

25. A surgical device comprising:
a leading end joint portion having a leading end grip portion;
a near-side joint portion having an operation portion; and
an arm portion storing a wire for linking motions of said leading end joint portion and said near-side joint portion,
wherein said leading end joint portion is moved downward and upward by operating said operation portion upward and downward around said near-side joint portion, and said leading end joint portion is moved rightward and leftward by operating said operation portion leftward and rightward, thereby making said leading end joint portion execute a swing motion, and
wherein said leading end grip portion is opened and closed by opening and closing said operation portion.

26. A surgical device as claimed in claim 25, wherein said operating portion is constituted by a pair of members capable of rotationally opening and closing only by an operation of the inserted finger and capable of holding said surgical device.
